(19) United States
(12) Patent Application Publication
(10) Pub. No.: US 2025/0231165 A1
(43) Pub. Date: Jul. 17, 2025

(54) DRUG PRODUCT SURROGATE SOLUTIONS

(71) Applicant: ModernaTX, Inc., Cambridge, MA (US)

(72) Inventors: Johnathan Goldman, Cambridge, MA (US); Satish K. Singh, Cambridge, MA (US); Anton Zverev Cambridge, Cambridge, MA (US); Michael H. Smith, Needham, MA (US)

(73) Assignee: ModernaTX, Inc., Cambridge, MA (US)

(21) Appl. No.: 18/703,189

(22) PCT Filed: Oct. 20, 2022
(86) PCT No.: PCT/US2022/047288

§ 371 Date: Apr. 19, 2024

Related U.S. Application Data
(60) Provisional application No. 63/257,779, filed on Oct. 20, 2021.

(51) Int. Cl. G01N 33/15; C12Q 1/18

(52) U.S. Cl. CPC G01N 33/15; C12Q 1/18

(57) ABSTRACT

Provided herein are methods and compositions of a surrogate mRNA drug product. The surrogate mRNA drug product is made up of a physical property surrogate, a visual property surrogate, and a chemical property surrogate. The surrogate mRNA drug product has at least one quantitative or qualitative property such as viscosity, turbidity, density, and surface tension that is comparable to and matches the same property of a mRNA drug product comprised of mRNA in a lipid nanoparticle (LNP). The surrogate mRNA drug product, however, does not comprise mRNA or LNP.

### RELATED APPLICATIONS

This application claims the benefit under 35 U.S.C. 119 (e) of U.S. provisional application No. 63/257,779 filed Oct. 20, 2021 which is incorporated by reference herein in its entirety.

### BACKGROUND

Messenger RNA (mRNA) is an emerging therapeutic platform offering a variety of drug product (DP) advantages including potency and programmability. mRNA-based medicines can be designed to promote the synthesis of a therapeutic protein or antigen in patients or provide a means of gene replacement. mRNA Lipid Nanoparticle (LNP) DPs have unique visual and physical properties, which can be measured qualitatively and quantitatively using a variety of methods.

### SUMMARY

Provided herein are surrogate solutions for lipid nanoparticle and/or mRNA drug products and methods of use thereof.

A composition is provided, in some aspects, that does not contain lipid nanoparticle (LNP) and/or mRNA and that has one or more physical property and/or one or more visual property that is substantially the same as a composition comprising LNP and/or mRNA.

In some embodiments the composition comprising the LNP and/or mRNA comprises a LNP encapsulating the mRNA. In some embodiments the composition comprising the mRNA is a mRNA drug product.

In some embodiments the physical property is viscosity, density, surface tension, propensity to foam, or a combination thereof.

In some embodiments the visual property is clarity, hue, or a combination thereof.

In some embodiments the chemical property is microbial growth.

In some embodiments the composition comprises a viscosity agent, a density agent, a surface tension agent, an anti-foaming agent, an antimicrobial agent, a turbidity agent, a coloring agent, or a combination thereof. In some embodiments the viscosity agent comprises a sugar, a polysaccharide, an alcohol, a polymer, polyethylene glycol (PEG), bovine serum albumin, or a combination thereof. In some embodiments the density agent comprises a sugar, a salt, an alcohol, or a combination thereof. In some embodiments, the density agent comprises a polysaccharide, such as cellulose or starch or any chemically modified derivative thereof. In some embodiments the surface tension agent comprises an anionic surfactant, a cationic surfactant, a non-ionic surfactant, or combinations thereof. In some embodiments the anti-foaming agent comprises a silicone, a fatty acid-ester, an ether, an alcohol, a combination of an ether and a polyether, or a combination thereof. In some embodiments the antimicrobial agent comprises a quaternary ammonium compound, an alkyl paraben, an alkyl/aryl alcohol, an alkyl/aryl acid and salt, a biguanide, an organic mercurial, a phenol, a formaldehyde releaser, an azide, or a combination thereof. In some embodiments the clarity agent comprises a particle, a bead, a sphere, a colloidal suspension, or combinations thereof. In some embodiments the coloring agent comprises an organic dye, an inorganic dye, or a combination thereof. In some embodiments the composition has substantially the same viscosity, density, surface tension, clarity, and hue as the composition comprising the mRNA.

In other aspects, a composition is provided, wherein the composition comprises a surrogate LNP and/or mRNA drug product comprised of a physical property surrogate, a chemical property surrogate and/or a visual property surrogate, wherein the surrogate LNP and/or mRNA drug product has at least one quantitative or qualitative property selected from the group consisting of viscosity, turbidity, density, and surface tension that is substantially the same as a drug product comprised of mRNA in a LNP, and wherein the surrogate drug product does not comprise mRNA or LNP.

A method for assessing a physical, chemical and/or visual property of an LNP and/or mRNA drug product is provided in other aspects. The method involves identifying a physical, chemical and/or a visual property of the composition of any one of the preceding claims, and determining that the physical, chemical and/or visual property of the LNP and/or mRNA drug product is within a pharmaceutically acceptable range.

In other aspects a method of calibrating an instrument for determining a physical, chemical and/or visual property of a LNP and/or mRNA drug product, the method comprising performing a measurement on the composition disclosed herein with the instrument, and optionally modifying one or more settings of the instrument. In some embodiments the method involves measuring the physical, chemical and/or visual property of the LNP and/or mRNA drug product. In some embodiments the instrument measures viscosity, density, surface tension, clarity, or hue.

A container comprising a vial or syringe is provided in other aspects. The container houses a surrogate mRNA drug product comprised of a physical property surrogate, a chemical property surrogate and/or a visual property surrogate and wherein the container does not comprise a lipid nanoparticle (LNP) or mRNA. In some embodiments the surrogate mRNA drug product comprises the composition disclosed herein.

Each of the features can encompass various embodiments of the invention. It is, therefore, anticipated that each of the elements involving any one feature or combinations of features can be included.

### DETAILED DESCRIPTION

The present disclosure relates to surrogate solutions for lipid nanoparticles and/or mRNA drug products (also referred to herein as surrogate solutions) which can be used to replace drug solutions in specific non-therapeutic situations. Specifically, surrogate solutions having defined properties mimic mRNA drug products. The surrogate solutions may be used in place of mRNA drug products in a number of instances. In some instances, the terms “surrogate LNP” and “LNP surrogate solution” may be used interchangeably.

Recently it has been demonstrated that mRNA formulated in lipid nanoparticles is an effective platform for delivering proteins to a subject. The production of a safe and effective drug solution requires careful analysis of physical, visual, and chemical properties of the material. The physical, visual, and chemical properties of mRNA drug products have been determined qualitatively and quantitatively, using methods including, but not limited to, viscometry, densiometry, turbidity, and spectrophotometry. Under conditions when mRNA drug supply is low or biological activity is irrelevant, the replacement of actual drug product with a surrogate is desirable. However, development of a surrogate having properties to sufficiently mimic a mRNA lipid nanoparticle drug product have been challenging.

According to aspects of the disclosure, surrogate solutions have been developed which closely match the physical, visual, and/or chemical properties of a composition comprising lipid particles such as lipid nanoparticles (LNPs). In some embodiments LNPs comprise (e.g., encapsulate) a nucleic acid, such as DNA and/or RNA (e.g., mRNA or siRNA). In some embodiments, the LNPs do not comprise a nucleic acid. Such compositions comprising a LNP can include a variety of components, such as ionizable lipids, neutral lipids, sterols, and/or a polyethylene glycol (PEG)-lipid. In some embodiments, the composition can contain lipid nanoparticles, liposomes, lipoplexes, lipid bilayers, or other lipid carriers. In some embodiments, the composition comprising a LNP which matches the surrogate solution is a LNP drug product (e.g., a mRNA drug product), which is a composition suitable for administration to an animal (e.g., human). In some embodiments, drug products have been approved by one or more regulatory agencies. In some embodiments the surrogate solutions have one or more properties that are “sufficiently similar” one or more properties of a LNP drug product.

In some embodiments, the surrogate solution does not contain a nucleic acid (e.g., mRNA). In some embodiments, the surrogate solution does not contain an ionizable lipid. In some embodiments, the surrogate solution does not contain a neutral lipid. In some embodiments, the surrogate solution does not contain a sterol. In some embodiments, the surrogate solution does not contain a PEG lipid. In some embodiments, the surrogate solution does not contain a LNP, a liposome, a lipoplex, and/or a lipid bilayer.

Drug products with lipid carriers typically appear as a cloudy, white solution suspending subvisible particles comprised of lipid-based transfection reagents. Lipid-based transfection reagents can encapsulate different therapeutic species including, for example, mRNA. mRNA drug products have varying physical properties owing to the lipids and the buffer components. The drug product generally has a water-like density and viscosity, but a low surface tension. The surrogate solutions can mimic the physical, visual, and chemical properties of mRNA drug products through their chemical composition. Methods for the quantification of drug product physical, visual, and chemical properties are also described. These methods can be used to qualify surrogate solution formulations by way of how closely they mimic the physical, visual, and chemical qualities of mRNA drug products.

In some aspects, a surrogate nucleic acid (e.g., mRNA) drug product is provided. The surrogate mRNA drug product can be comprised of a physical property surrogate, a visual property surrogate, and a chemical property surrogate.

A visual property surrogate is a component of a solution which has a property which mimics a visual property of the nucleic acid solution. A visual property is a property that is assessed through visible observation, by quantitative and/or qualitative means. Visual properties of nucleic acid solutions include, for instance, clarity (measured by turbidity and/or visual comparison) and hue (color, measured by absorbance and/or visual comparison). In nucleic acid solutions, such properties are influenced by the excipients (e.g. colored additives) and the size and concentration of particles in the solution.

A physical property surrogate is a component of a solution which has a property which mimics a physical property of the nucleic acid solution. A physical property is a property that is assessed through measurement, by quantitative and/or semi-quantitative means. Physical properties of nucleic acid solutions include, for instance, density, viscosity, surface tension, and propensity to foam. In nucleic acid solutions, such properties are influenced by the excipients (e.g. sugars) and lipids.

A chemical property surrogate is a component of a solution which has a property which mimics a chemical property of the nucleic acid solution. A chemical property is a property that is inherent to a solution and relates to its chemical reactivity. Chemical properties of nucleic acid solutions include, for instance, antimicrobial activity. In nucleic acid solutions, such properties are influenced by the nucleic acids (e.g. mRNA) and lipids.

A property of a surrogate solution mimics a property of a drug solution when that property falls within an acceptable range of a quantitative or qualitative measurement when measured under similar or same conditions such as temperature and time. Acceptable ranges for the various quantitative or qualitative measurements associated with mRNA drug product properties are determined using a variety of methods, including many of the methods disclosed herein. A property which mimics the drug property is determined relative to the values of the specific LNP composition (e.g., drug product) to which it is being compared. Exemplary ranges are provided throughout the disclosure.

The surrogate solution is typically comprised of a buffer matrix that includes agents that impart particular visual, chemical, and/or physical properties upon the solution. In some embodiments, the buffer matrix is an aqueous solution. In some embodiments, the buffer matrix contains 80% or more of water, such as 81%, 82%, 83%, 84%, 85%, 86%, 87%, 88%, 89% 90%, 91%, 92%, 93%, 94%,95%, or more of water. For instance, such agents can include a viscosity agent, a density agent, a surface tension agent, an anti-foaming agent, an antimicrobial agent, a turbidity agent, a coloring agent, or a combination thereof. In some embodiments, an agent can provide dual-functionality. For example, in some embodiments, a viscosity agent can also be a density agent, a surface tension agent, etc.

Exemplary physical properties of a mRNA drug product which have been characterized are shown in Table 1. Corresponding physical properties of a surrogate solution having properties which mimic the exemplary mRNA drug product are also shown in Table 1.

TABLE 1
Comparison of physical properties of mRNA
drug products to surrogate solutions.

| Property | mRNA Drug Product | Surrogate |
|---|---|---|
| Viscosity at 5000 s⁻¹ (cP) | 1.0-1.1 | 1.1 |
| Surface Tension (mN/m) | 1.0-1.1 | 1.2 |
| Density (g/mL) | 1.0 | 1.0 |
| Propensity to Foam | Minimal foaming | Minimal foaming |
| | when shaken | when shaken |

Note:
Properties are measured at room temperature unless stated otherwise. Quantitative values are normalized to a value of 1 for the mRNA Drug Product.

Exemplary visual properties of two mRNA drug products which have been characterized are shown in Tables 2 and 3. Corresponding visual properties of surrogate solutions having properties which mimic the exemplary mRNA drug products are also shown in Tables 2 and 3.

TABLE 2
Replicate 1 data comparing visual properties
of mRNA drug products to surrogate solutions.

| | mRNA Drug | Surrogate, | mRNA Drug | Surrogate, |
|---|---|---|---|---|
| Property | Product 1 | Batch 1 | Product 2 | Batch 2 |
| Turbidity (NTU) | 1.0-2.0 | 1.2-1.3 | 1.5-3.0 | 1.8-1.9 |
| Absorbance at | 1.0-1.5 | 1.0-1.1 | 1.3-3.0 | 2.0-2.1 |
| 400 nm (AU) | | | | |
| Appearance | White to | White to | White to | White to |
| | off-white | off-white | off-white | off-white |

Note:
Quantitative values are normalized to a value of 1 for the mRNA Drug Product.

TABLE 3
Replicate 2 data comparing visual properties
of mRNA drug products to surrogate solutions.

| | mRNA | | mRNA | | mRNA | |
|---|---|---|---|---|---|---|
| | Drug | Surrogate | Drug | Surrogate | Drug | Surrogate |
| Property | Product 3 | Batch 3 | Product 4 | Batch 4 | Product 5 | Batch 5 |
| Turbidity | 1.0-3.0 | 1.6-1.8 | 2.0-4.0 | 2.4-2.6 | 3.0-6.0 | 3.6-3.8 |
| (NTU) | | | | | | |
| Absorbance at | 1.0-2.1 | 1.5-1.8 | 2.4-3.6 | 2.4-2.7 | 3.0-7.2 | 4.8-5.4 |
| 400 nm (AU) | | | | | | |
| Appearance | White to | White to | White to | White to | White to | White to |
| | off-white | off-white | off-white | off-white | off-white | off-white |

Note:
Quantitative values are normalized to a value of 1 for the mRNA Drug Product.

Exemplary chemical properties of a mRNA drug product which have been characterized are shown in Table 4. Corresponding chemical properties of a surrogate solution having properties which mimic the exemplary mRNA drug product are also shown in Table 4.

TABLE 4
Comparison of chemical properties of mRNA
drug products to surrogate solutions.

| Property | mRNA Drug Product | Surrogate |
|---|---|---|
| Antimicrobial Activity | Passes antimicrobial | Includes antimicrobial |
| | effectiveness test | preservative |

### Visual Property Surrogates

A visual property surrogate is a component of a solution which has a property which mimics a visual property of the nucleic acid solution. A visual property is a property that is assessed through visible observation, by quantitative and/or qualitative means. Visual properties of nucleic acid solutions include, for instance, clarity and hue. The visual properties of mRNA drug product are affected by LNP size, subvisible particles, and concentration. These visual properties may vary over the acceptable specification ranges for product attributes. For example, a mRNA drug product specification that allows for varying particle size and varying concentration may have substantial differences in visual properties for different drug product batches at different ends of the size and concentration ranges.

A surrogate having properties which mimic the properties of the drug product can be selected to capture the appearance of a typical representative batch of drug product. For example, visual properties of an exemplary mRNA drug product are shown in Table 2. Corresponding visual properties of surrogate solutions having properties which mimic the exemplary mRNA drug product are shown in Table 2.

### Clarity

In some embodiments, the visual property is clarity. Clarity relates to the cloudiness or haziness in a composition, and it is sometimes referred to using the terms opacity, opalescence, or turbidity.

Clarity may be assessed quantitatively using, for instance, a turbidimeter and/or a spectrophotometer. A turbidimeter is a measuring tool which comprises at least one light source and at least one light detector positioned at a 90 degree angle relative to the light source and is configured in such a way that it makes it possible to measure the scattering of light in a sample or a reaction mixture. Either nephelometric turbidity or ratio turbidity may be employed. A spectrophotometer is a measuring unit which comprises at least one light source and at least one light detector positioned at a 180 degree angle relative to the light source and is configured in such a way that it makes it possible to measure the absorbance of light in a sample or a reaction mixture. Typically, the wavelength of the light emitted by the light source is selected in such a way that it is scattered and/or absorbed by macromolecules to be detected in the sample, for example particles and particle aggregates.

In some embodiments, the composition (such as a LNP drug product and/or a LNP surrogate solution) comprises a turbidity of from about 50 NTU to about 1,000 NTU, about 100 NTU to about 1,000 NTU, about 150 NTU to about 1,000 NTU, about 200 NTU to about 1,000 NTU, about 250 NTU to about 1,000 NTU, about 300 NTU to about 1,000 NTU, about 350 NTU to about 1,000 NTU, about 400 NTU to about 1,000 NTU, about 450 NTU to about 1,000 NTU, about 500 NTU to about 1,000 NTU, about 550 NTU to about 1,000 NTU, about 600 NTU to about 1,000 NTU, about 650 NTU to about 1,000 NTU, about 700 NTU to about 1,000 NTU, about 750 NTU to about 1,000 NTU, about 800 NTU to about 1,000 NTU, about 850 NTU to about 1,000 NTU, about 900 NTU to about 1,000 NTU, about 950 NTU to about 1,000 NTU, about 1,000 NTU to about 2,000 NTU, about 1,100 NTU to about 2,000 NTU, about 1,200 NTU to about 2,000 NTU, about 1,300 NTU to about 2,000 NTU, about 1,400 NTU to about 2,000 NTU, about 1,500 NTU to about 2,000 NTU, about 1,600 NTU to about 2,000 NTU, about 1,700 NTU to about 2,000 NTU, about 1,800 NTU to about 2,000 NTU, or about 1,900 NTU to about 2,000 NTU. In some embodiments the turbidity is from about 200 to about 800 NTU, about 200 to about 600 NTU, about 300 to about 500 NTU, about 200 to about 400 NTU, about 300 to about 600 NTU, about 240 to about 260 NTU, about 360 to about 380 NTU, or about 400 to about 500 NTU. In some embodiments, the composition comprises a turbidity of about 50 NTU, about 100 NTU, about 150 NTU, about 200 NTU, about 250 NTU, about 300 NTU, about 350 NTU, about 400 NTU, about 450 NTU, about 500 NTU, about 550 NTU, about 600 NTU, about 650 NTU, about 700 NTU, about 750 NTU, about 800 NTU, about 850 NTU, about 900 NTU, about 950 NTU, or about 1000 NTU. In some embodiments, the turbidity of the composition is represented as a value that is normalized to a reference sample (e.g., a standard solution of mRNA drug product). In some embodiments, the turbidity of a reference sample may be given a value of 1.0 NTU and the composition (such as a LNP drug product and/or a LNP surrogate solution) comprises a turbidity value of 0.5 NTU to about 10.0 NTU, about 1.0 NTU to about 10.0 NTU, about 1.5 NTU to about 10.0 NTU, about 2.0 NTU to about 10.0 NTU, about 2.5 NTU to about 10.0 NTU, about 3.0 NTU to about 10.0 NTU, about 3.5 NTU to about 10.0 NTU, about 4.0 NTU to about 10.0 NTU, about 4.5 NTU to about 10.0 NTU, about 5.0 NTU to about 10.0 NTU, about 5.5 NTU to about 10.0 NTU, about 6.0 NTU to about 10.0 NTU, about 6.5 NTU to about 10.0 NTU, about 7.0 NTU to about 10.0 NTU, about 7.5 NTU to about 10.0 NTU, about 8.0 NTU to about 10.0 NTU, about 8.5 NTU to about 10.0 NTU, about 9.0 NTU to about 10.0 NTU, about 9.5 NTU to about 10.0 NTU, about 10.0 NTU to about 20.0 NTU, about 11.0 NTU to about 20.0 NTU, about 12.0 NTU to about 20.0 NTU, about 13.0 NTU to about 20.0 NTU, about 14.0 NTU to about 20.0 NTU, about 15.0 NTU to about 20.0 NTU, about 16.0 NTU to about 20.0 NTU, about 17.0 NTU to about 20.0 NTU, about 18.0 NTU to about 20.0 NTU, or about 19.0 NTU to about 20.0 NTU. In some embodiments the turbidity is from about 2.0 to about 8.0 NTU, about 2.0 to about 6.0 NTU, about 3.0 to about 5.0 NTU, about 2.0 to about 4.0 NTU, about 3.0 to about 6.0 NTU, about 2.4 to about 2.6 NTU, about 3.6 to about 3.8 NTU, or about 4.0 to about 5.0 NTU. In some embodiments, the composition comprises a turbidity of about 0.5 NTU, about 1.0 NTU, about 1.5 NTU, about 2.0 NTU, about 2.5 NTU, about 3.0 NTU, about 3.5 NTU, about 4.0 NTU, about 4.5 NTU, about 5.0 NTU, about 5.5 NTU, about 6.0 NTU, about 6.5 NTU, about 7.0 NTU, about 7.5 NTU, about 8.0 NTU, about 8.5 NTU, about 9.0 NTU, about 9.5 NTU, or about 10.0 NTU. In some embodiments the composition comprises a turbidity within a range in which the lower and upper limit are any combination of the preceding values.

In some embodiments, the composition (such as a LNP drug product and/or a LNP surrogate solution) comprises an absorbance measurement. Absorbance (measured by spectrophotometry) can be measured at any wavelength in the near-UV, visible, or near-IR range (approximately 300 nm to 800 nm). The absorbance values of the LNP drug product and/or LNP surrogate solution will vary depending on the wavelength that is used, but generally the values will range from 0 to 4 Absorbance Units.

In some embodiments, the composition comprises a clarity agent. For instance, a mRNA surrogate solution can comprise a clarity agent in an amount sufficient to provide a turbidity measurement that is sufficiently similar to the turbidity measurement of a mRNA drug product. “Sufficiently similar” in this context indicates that the measurement is within a pre-defined difference (for instance, 20%) of the value expected for the mRNA drug product, additionally considering a reasonable lot-to-lot variability observed in the mRNA drug product.

Exemplary clarity agents include particles, beads, spheres, colloidal suspensions, or combinations thereof. Some embodiments comprise micro- and/or nano-particles, beads, and/or spheres. In some embodiments, the clarity agent comprises a metal or metalloid oxide, such as gold, silver, iron oxide, aluminum oxide, magnesium, magnesium oxide and other alkaline-earth oxides, zirconium oxides, cerium oxides, silicon oxides, or titanium oxides. In some embodiments, the clarity agent comprises a polymer, such as polystyrene, polypropylene, polyethylene, polymethyl methacrylate (PMMA), etc. In some embodiments, the clarity agent comprises a surface modification, such as addition of one of more carboxyl groups, hydroxyl groups, etc. In some embodiments, the clarity agent comprises a colloidal suspension, such as formazine, etc.

In some embodiments, the clarity agent has a size of greater than 1 micron, or about 1-10 microns. Alternatively, the clarity agent may have a smaller size and may be a nanoparticle. Nanoparticles typically have a size of less than 1 micron in diameter. In some embodiments a nanoparticle is 1 nm-10 nm, 1 nm-50 nm, 1 nm-100 nm, 1 nm-250 nm, 1 nm-500 nm, 1 nm-750 nm, 1 nm-999 nm, 10 nm-50 nm, 10 nm-100 nm, 50 nm-200 nm, 80 nm-160 nm, 80 nm-150 nm, 200 nm-400 nm, 300 nm-600 nm, 240 nm-260 nm, 360 nm-380 mn, 10 nm-250 nm, 10 nm-500 nm, 10 nm-750 nm, or 10 nm-999 nm.

In some embodiments, the clarity agent may be a polymeric particle. Polymeric materials are known and available for use in the production of particles. The polymers used to make polymeric particles may be biocompatible and/or biodegradable polymers. The polymers in some embodiments are homopolymers, copolymers, or a combination thereof. Biodegradable or biocompatible polymers can include one or more of the following: polyesters (poly(caprolactone); poly(hydroxy acids), such as poly(lactic acid), poly(glycolic acid), and poly(lactic acid-co-glycolic acids); polyhydroxyalkanoates, such as poly(3-hydroxybutyrate) and poly(4-hydroxybutyrate)); polyanhydrides (poly(fumaric-co-sebacic acid), polysebacic acid, polyfumaric acid); poly(orthoesters); hydrophobic polypeptides; hydrophobic polyethers, such as poly(propylene oxide); poly(phosphazenes), polyesteramides, poly(alkylene alkylates), polyether esters, polyacetals, polycyanoacrylates, polyketals, polyhydroxyvalerates, polyalkylene oxalates, polyalkylene succinates, mixtures, and copolymers thereof. In some embodiments, the contain lactic acid units, such as poly-L-lactic acid, poly-D-lactic acid, poly-D, L-lactic acid, poly-L-lactide, poly-D-lactide, and poly-D, L-lactide (PLA). Poly(lactic acid-co-glycolic acid) and poly(lactide-co-glycolide) polymers, characterized by the ratio of lactic acid: glycolic acid, are referred to as PLGA polymers.

In some embodiments the polymeric particle comprises polystyrene, polypropylene, polyethylene, or PMMA. The polymeric particle, in some embodiments, has a surface modification. The surface may be modified to provide altered or enhanced particulate properties. For instance, the surface may be modified with a carboxyl or hydroxyl group.

In some embodiments the LNP composition contains a mixture of clarity agents (e.g., polystyrene nanoparticles) selected to match the clarity of the drug product. For example, polystyrene (PS) beads with various particle sizes (e.g., 100 nm and 200 nm particle sizes) may be used to match the turbidity and absorbance of a mRNA drug product. If two or more particle sizes are used, they can be mixed in any suitable proportion.

In some embodiments, the composition comprises a concentration of clarity agent in the range of about 0.001% to 0.1%. Generally lower concentration may be needed for larger polymeric particles. For example, up to about 0.04% 100 nm PS beads, up to about 0.02% 200 nm PS beads, or up to about 0.01% 500 nm PS beads may be used.

### Hue/Color

In some embodiments, the visual property is hue or color. The hue or color can be determined by any suitable method, such as spectrophotometric absorbance or visual inspection. The hue/color can be determined using absorbance since certain wavelengths or absorbance measurements are indicative of different hues/colors. Substances that look a certain color will typically have a low absorbance at that corresponding wavelength. For example, a substance that looks red will have very low absorbance at wavelengths corresponding to red (600-700 nm). In some embodiments, the composition (such as a LNP drug product and/or a LNP surrogate solution) comprises a hue or color that is white. In some embodiments, the hue or color is off-white. In some embodiments, the hue or color is yellow, blue, orange, red, and brown. In some embodiments, the composition comprises a coloring agent, such as a dye (e.g., an organic dye, inorganic dye, or combination thereof). For instance, a mRNA surrogate solution can comprise a coloring agent in an amount sufficient to provide a hue or color that is substantially the same as the hue or color of a mRNA drug product.

In some embodiments, the dye is an organic dye, such as an azo dye, anthraquinone dye, or carotene. In some embodiments, the dye is an inorganic dye, such as a transition metal salt or coordination compound. In some embodiments the dye may be any of a heteroaromatic dye, an azo dye; Congo Red; curcumin analog; X-34 (1,4-bis(3-carboxy-4-hydroxyphenylethenyl)-benzene); thioflavin S; thioflavin T; Nile Red; acridine orange; amino-8-napthalene sulfonate (ANS); bis-ANS; 4-(dicyanovinyl)-julolidine (DCVJ); A01987 (oxazine dye); fluorescent styryl dyes; BF-168: (6-2-Fluoroethoxy)-2-[2-(4-methylaminophenil) ethenyl] benzoxazole; BSB: (trans,trans)-1-bromo-2,5-bis-(3-hydroxycarbonyl-4-hydroxy) styrylbenzene; quinilinehydrazone compounds (e.g., 4-methyl-7-methoxy-2-(4-quinolylmethylenehydrazino) quinoline); [11C]-PIB: N-methyl [11C] 2-(4′-methylaminophenyl-6-hydroxybenzathiazole); stilbenylbenzothiazole or stilbenylbenzothiazole derivative; common food dyes such as FD&C#2,3,4,5,6, 40 etc. or any combination thereof.

In some embodiments, the composition comprises a range of about 0 to 1% of coloring agent.

### Physical Property Surrogates

A physical property surrogate is a component of a solution which has a property which mimics a physical property of the nucleic acid solution. A physical property is a property that is assessed through measurement, by quantitative and/or semi-quantitative means. Physical properties of nucleic acid solutions include, for instance, density, viscosity, surface tension, and propensity to foam.

The physical property surrogate may be defined by the buffer matrix, which can contain one or more of a density agent, a viscosity agent, a surface tension agent, an anti-foaming agent, and a combination thereof. The buffer matrix in some embodiments comprises a sugar, a surfactant, and an anti-foaming agent.

### Density

In some embodiments, the physical property is density. Density may be measured using a density meter such as a densitometer.

In some embodiments, the composition (such as a LNP drug product and/or a LNP surrogate solution) has a density of about 0.25 g/mL or more, such as about 0.5 g/mL, about 0.75 g/mL, about 1 g/mL, about 1.25 g/mL, about 1.5 g/mL, about 1.75 g/mL, about 2 g/mL, about 3 g/mL, about 4 g/mL, or about 5 g/mL. In some embodiments the composition comprises a density within a range in which the lower and upper limit are any combination of the preceding values.

In some embodiments, the composition comprises a density agent. For instance, a mRNA surrogate solution can comprise a density agent in an amount sufficient to provide a density that is substantially the same as the density of a mRNA drug product.

Exemplary density agents include sugars, polysaccharides, salts, alcohols, polyethylene glycol (PEG), bovine serum albumin, or combinations thereof. In some embodiments, the density agent comprises a sugar, such as sucrose, trehalose, sorbitol, dextrose, fructose, mannitol or combination thereof. In some embodiments, the density agent comprises a polysaccharide, such as cellulose or starch or any chemically modified derivative thereof. In some embodiments, the density agent comprises a salt, such as an alkali or alkaline-earth chloride, bromide, carbonate, sulfate, or phosphate salt, or a combination thereof. In some embodiments, the density agent comprises an alcohol, such as ethanol, propanol, methanol, butanol, or any other alcohol (or any isomer of a particular alcohol) or a combination thereof.

In some embodiments, the composition comprises about 0, or 1, or 2, or 3, or 4, or 5, or 6, or 7, or 8, or 9, or 10, or 11, or 12, or 13, or 14, or 15, or 16, or 17, or 18, or 19 to 20% of density agent.

### Viscosity

In some embodiments, the physical property is viscosity. Viscosity can be measured using a viscometer or rheometer. A rheology meter, such as a viscometer or a rheometer, may be used to make a quantitative measurement of viscosity. Viscometers measure the viscosity and flow properties of a fluid. A rheometer measures the way a fluid responds to applied shear or stress, thus providing viscosity data as well as data on structural and elasticity features. Viscosity is generally reported at a pre-defined shear rate, for instance 100, 1000, 5000, or 10000 s-1.

In some embodiments, the composition (such as a LNP drug product and/or a LNP surrogate solution) has a viscosity at 5000 s-1 of about 0.25 cP or more, such as about 0.5 cP, about 0.75 cP, about 1.0 cP, about 1.25 cP, about 1.5 cP, about 1.75 cP, about 2 cP, about 3 cP, about 4 cP, or about 5 cP. In some embodiments, the composition (such as a LNP drug product and/or a LNP surrogate solution) has a viscosity at 5000 s-1 of about 10 cP or more, such as about 15 cP, about 20 cP, about 25 cP, about 26 cP, about 27 cP, about 28 cP, about 29 cP, about 30 cP or more. In some embodiments the composition comprises a viscosity within a range in which the lower and upper limit are any combination of the preceding values.

In some embodiments, the composition comprises a viscosity agent. For instance, a mRNA surrogate solution can comprise a viscosity agent in an amount sufficient to provide a viscosity that is substantially the same as the viscosity of a mRNA drug product.

Exemplary viscosity agents include sugars, polysaccharides, alcohols, polymers, bovine serum albumin, or combinations thereof. In some embodiments, the viscosity agent comprises a sugar, such as sucrose, trehalose, sorbitol, dextrose, fructose, mannitol, or combination thereof. In some embodiments, the viscosity agent comprises a polysaccharide, such as cellulose or starch or any chemically modified derivative thereof. In some embodiments, the viscosity agent comprises an alcohol, such as ethanol, propanol, methanol, butanol, or any other alcohol (or any isomer of a particular alcohol) or a combination thereof. In some embodiments, the viscosity agent comprises a polymer (e.g., a water-soluble polymer), such as PEG, PVP, PVA, PAA, or combinations thereof.

In some embodiments, the composition comprises about 0, or 1, or 2, or 3, or 4, or 5, or 6, or 7, or 8, or 9, or 10, or 11, or 12, or 13, or 14, or 15, or 16, or 17, or 18, or 19 to 20% of viscosity agent.

### Surface Tension

In some embodiments, the physical property is surface tension. Surface tension may be measured using a tensiometer or goniometer, which can provide an optical measurement of the surface tension and interfacial tension of a liquid.

In some embodiments, the composition (such as a LNP drug product and/or a LNP surrogate solution) has a surface tension of about 15 mN/m or more, such as about 20 mN/m, about 25 mN/m, about 30 mN/m, about 32 mN/m, about 35 mN/m, about 39 mN/m, about 40 mN/m, about 45 mN/m, about 50 mN/m, about 55 mN/m, about 60 mN/m or about 20 to 80 mN/m. In some embodiments the composition comprises a surface tension within a range in which the lower and upper limit are any combination of the preceding values.

In some embodiments, the composition comprises a surface tension agent. For instance, a mRNA surrogate solution can comprise a surface tension agent in an amount sufficient to provide a surface tension that is substantially the same as the surface tension of a mRNA drug product.

The components in the surrogate solution that provide altered surface tension include anionic surfactants, cationic surfactants, non-ionic surfactants, other surfactants, or combinations thereof. In some embodiments the surface tension agent is an anionic surfactant, such as an alkyl sulfate, and alkylbenzene sulfonate, or a combination thereof. In some embodiments, the surface tension agent is a cationic surfactant, such as a quaternary ammonium compound. In some embodiments, the surfactant is a non-ionic surfactant, such as an ethoxylated alcohol, an ethoxylated alkyl phenol, a fatty acid ester, triton X-100, or combinations thereof. In some embodiments, the surfactant is a silicon compound, a fluorinated compound, or a combination thereof.

In some embodiments, the composition comprises about 0 to 1% of surface tension agent.

### Propensity to Foam

In some embodiments, the physical property is a propensity to foam. Propensity to foam may be assessed semi-quantitatively or qualitatively.

In some embodiments, the composition (such as a LNP drug product and/or a LNP surrogate solution) has a no, a minimal, a medium, or a high propensity to foam when shaken. The propensity to foam may be assessed by the amount of foam generated during a pre-defined shaking or agitation motion, as well as the time it takes the foam to dissipate.

In some embodiments, the composition comprises an antifoaming agent. For instance, a mRNA surrogate solution can comprise an antifoaming agent in an amount sufficient to provide a propensity to foam that is sufficiently similar to the propensity to foam of a mRNA drug product.

In some embodiments, the antifoaming agent is a silicone, a fatty acid-ester, an ether, an alcohol, a combination of an ether and a polyether, or a combination thereof. In some embodiments, the silicone comprises dimethicone, polysiloxane, or a combination thereof. In some embodiments, the ester comprises glycol distearate, sorbitol laurate, or a combination thereof. In some embodiments, the ether comprises propylene glycol. In some embodiments, the polyether comprises polypropylene glycol. The anti-foaming agent may be an Antifoam 204 and 1,2-propanediol (propylene glycol).

In some embodiments, the composition comprises about 0, or 1, or 2, or 3, or 4 to 5% of antifoaming agent.

### Chemical Property Surrogates

A chemical property surrogate is a component of a solution which has a property which mimics a chemical property of the nucleic acid solution. A chemical property is a property that is inherent to a solution and relates to its chemical reactivity. Chemical properties of nucleic acid solutions include, for instance, antimicrobial activity. The chemical property surrogate may be defined by the buffer matrix, which can contain an antimicrobial agent.

### Antimicrobial Activity

In some embodiments, the chemical property is antimicrobial activity, i.e. microbial growth prevention. Antimicrobial activity may be measured using an antimicrobial effectiveness test.

In some embodiments, the composition (such as a LNP drug product and/or a LNP surrogate solution) passes an antimicrobial effectiveness test. Examples of antimicrobial tests may include, but are limited to, mycoplasma testing, endotoxin testing, microbial immersion studies, microbial limits testing, 16S rRNA gene long sequencing, 25S rRNA gene or ITS1/ITS2 partial sequencing.

In some embodiments, the composition comprises an antimicrobial agent. For instance, a mRNA surrogate solution can comprise an antimicrobial agent in an amount sufficient to prevent microbial growth to a sufficiently similar extent as a mRNA drug product.

In some embodiments the antimicrobial agent comprises a quaternary ammonium compound, an alkyl paraben, an alkyl/aryl alcohol, an alkyl/aryl acid and salt, a biguanide, an organic mercurial, a phenol, a formaldehyde releaser, an azide, or a combination thereof. In some embodiments, the quaternary ammonium compounds comprises benzalkonium chloride, cetrimonium bromide, or a combination thereof. In some embodiments, the alkyl paraben comprises methylparaben, ethylparaben, propylparaben, butylparaben, or a combination thereof. In some embodiments, the alkyl/aryl alcohol comprises benzyl alcohol, steryl alcohol, or a combination thereof. In some embodiments, the alkyl/aryl acid and salt comprises benzoic acid, sorbic acid, or a combination thereof. In some embodiments, the biguanide comprises chlorhexidine. In some embodiments, the organic mercurial comprises thimerosal. In some embodiments, the phenol comprises m-cresol, phenol, or a combination thereof. In some embodiments, the formaldehyde releaser comprises imidurea, bronopol, or a combination thereof. In some embodiments, the azide comprises sodium azide.

In some embodiments, the composition comprises about 0 to 2% of antimicrobial agent.

After all the quantitative methods have been performed, an additional way to ensure the surrogate solution is “sufficiently similar” to the drug product is to perform a randomized visual comparison with several operators/scientists. If operators are unable to consistently distinguish surrogate vials from drug product vials in a visual inspection, the surrogate is deemed “sufficiently similar”.

The surrogate, in some embodiments is composed of chemically unreactive entities and remains stable for long periods of time at room temperature or refrigerated temperatures, e.g. at least 1 year. Stable refers to a minimal change to visual/physical/chemical properties over time. The stability of the surrogate is an additional benefit that may extend its utility over drug product.

### Surrogate Applications

LNP surrogates have broad utility across a variety of fields. For instance, drug products must be tested using a variety of instruments to confirm that the products comply with various quality attributes. Setting up and calibrating the instruments often uses significant amount of drug product, wasting precious resources and increasing costs of drug development. Thus, in some embodiments, analytical instruments are calibrating using nucleic acid (e.g., mRNA) surrogate solutions. In some embodiments, such analytical instruments evaluate physical or visual property of a composition, such as density, viscosity, surface tension, propensity to foam, microbial growth, clarity, and/or hue.

Similarly, testing processing, handling, and/or shipping conditions associated with a nucleic acid composition (e.g., drug product) often uses significant amounts of the nucleic acid composition. Thus, in some embodiments, surrogate nucleic acid solutions are used to test processing, handling, and/or shipping conditions, which can eventually be used with a nucleic acid drug product.

Some embodiments comprise conducting mixing simulations with the nucleic acid surrogate solution. In some embodiments, the mixing simulations include performing dilution experiments, compounding experiments, or both to determine conditions for use with the nucleic acid containing composition (e.g., drug product).

In some embodiments, surrogate solutions for mRNA drug products can be used when obtaining a large volume of real drug product is expensive or logistically challenging, and when macroscopic visual and/or physical properties are deemed important. For instance, the surrogate may be used for training, e.g. demonstrations of manufacturing operations or clinical handling. Another use of the surrogate is to aid in visual inspection development, e.g. vials or pre-filled syringes are filled with surrogate and seeded with particulate defects to make defect kits. The surrogate mRNA drug product may also be used for optimizing manufacturing conditions (e.g., mixing speed, mixing duration, etc.). The surrogate mRNA drug product may even be used as memorabilia, e.g. vials or pre-filled syringes filled with surrogate as commemorative displays. Additionally, described are methods for characterizing the visual and physical properties of mRNA drug products and the application of said methods for qualifying surrogate solutions for LNP and/or mRNA drug products. In some embodiments the surrogate mRNA drug products incorporate visual properties which mimic those of a corresponding mRNA drug product. Visual properties include, for instance, one or more of clarity and hue. These types of visual properties may be measured using instruments that detect and quantify turbidity and/or absorbance. Quantitative measurements such as turbidity and absorbance can distinguish differences between samples that would not be noticed visually.

In some embodiments the surrogate mRNA drug products incorporate physical properties which mimic those of a corresponding mRNA drug product. Physical properties include, for instance, one or more of density, viscosity, surface tension, and propensity to foam., In some embodiments the surrogate mRNA drug products incorporate chemical properties which mimic those of a corresponding mRNA drug product. Chemical properties include, for instance, antimicrobial activity.

Thus, one aspect of the present disclosure relates to methods for visual and physical characterization of surrogate mRNA drug products. In embodiments of the present disclosure, the said methods are also applicable for qualifying surrogate solutions by way of their ability to mimic the properties of mRNA drug products.

In some aspects, the surrogate mRNA drug product is useful in methods for detecting defects in drug product. In some aspects, the surrogate mRNA drug product may include a particulate defect. The particulate defect may cause distinct visual or physical properties which fall outside of a normal range for mRNA drug product. These surrogates can be used to detect defects in mRNA drug products. They can be used for instance in defect detection kits.

In some embodiments, the surrogate mRNA does not include mRNA or ionizable lipids, but nevertheless mimics a mRNA in a LNP in terms of visual and physical properties. The following examples are set forth. The examples are offered to illustrate the methods, compounds, and pharmaceutical compositions provided herein and are not to be construed in any way as limiting their scope.

### EXAMPLES

### Example 1: Preparation and Analysis of mRNA Drug Surrogate Solutions

This example describes preparation and analysis of mRNA drug surrogate solutions. The components listed in Table 5 are combined in the amounts listed in the table.

TABLE 5
Chemical composition of example surrogates.

| | Content (%) | | |
|---|---|---|---|
| Chemical Name | Surrogate 1 | Surrogate 2 | Surrogate 3 |
| Sucrose | 9 | 9 | 9 |
| Polystyrene beads, | 0.015 | 0.03 | 0.03 |
| 0.1 μm | | | |
| Polystyrene beads, | 0.005 | 0.01 | 0.01 |
| 0.2 μm | | | |
| Polystyrene beads, | 0 | 0 | 0.01 |
| 0.5 μm | | | |
| Triton X-100, | 0.01 | 0.01 | 0.01 |
| Reduced | | | |
| 1,2-propanediol | 2.0 | 2.0 | 2.0 |
| Antifoam 204 | 0.04 | 0.04 | 0.04 |
| Sodium Azide | 0.02 | 0.02 | 0.02 |
| Water | Balance | Balance | Balance |

### Equivalents and Scope

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in some embodiments, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in some embodiments, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. Each possibility represents a separate embodiment of the present invention.

It should be understood that, unless clearly indicated to the contrary, the disclosure of numerical values and ranges of numerical values in the specification includes both i) the exact value(s) or range specified, and ii) values that are “about” the value(s) or ranges specified (e.g., values or ranges falling within a reasonable range (e.g., about 10% similar)) as would be understood by a person of ordinary skill in the art.

It should also be understood that, unless clearly indicated to the contrary, in any methods disclosed herein that include more than one step or act, the order of the steps or acts of the method is not necessarily limited to the order in which the steps or acts of the method are disclosed.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A composition that does not contain lipid nanoparticle (LNP) and/or mRNA and that has one or more physical property and/or one or more visual property that is substantially the same as a composition comprising LNP and/or mRNA.

2. The composition of claim 1, wherein the composition comprising the LNP and/or mRNA comprises a LNP encapsulating the mRNA.

3. The method of any one of the preceding claims, wherein the composition comprising the mRNA is a mRNA drug product.

4. The composition of claim 1, wherein the physical property is viscosity, density, surface tension, propensity to foam, or a combination thereof.

5. The composition of any one of the preceding claims, wherein the visual property is clarity, hue, or a combination thereof.

6. The composition of claim 1, wherein the chemical property is microbial growth.

7. The composition of any one of the preceding claims, wherein the composition comprises a viscosity agent, a density agent, a surface tension agent, an anti-foaming agent, an antimicrobial agent, a turbidity agent, a coloring agent, or a combination thereof.

8. The composition of claim 7, wherein the viscosity agent comprises a sugar, an alcohol, a polymer, or a combination thereof.

9. The composition of claim 7 or 8, wherein the density agent comprises a sugar, a polysaccharide, a salt, an alcohol, polyethylene glycol, bovine serum albumin, or a combination thereof.

10. The composition of any one of claims 7-9, wherein the surface tension agent comprises an anionic surfactant, a cationic surfactant, a non-ionic surfactant, or combinations thereof.

11. The composition of any one of claims 7-10, wherein the anti-foaming agent comprises a silicone, a fatty acid-ester, an ether, an alcohol, a combination of an ether and a polyether, or a combination thereof.

12. The composition of any one of claims 7-11, wherein the antimicrobial agent comprises a quaternary ammonium compound, an alkyl paraben, an alkyl/aryl alcohol, an alkyl/aryl acid and salt, a biguanide, an organic mercurial, a phenol, a formaldehyde releaser, an azide, or a combination thereof.

13. The composition of any one of claims 7-12, wherein the clarity agent comprises a particle, a bead, a sphere, a colloidal suspension, or combinations thereof.

14. The composition of any one of claims 7-13, wherein the coloring agent comprises an organic dye, an inorganic dye, or a combination thereof.

15. The composition of any one of the preceding claims, wherein the composition has substantially the same viscosity, density, surface tension, clarity, and hue as the composition comprising the mRNA.

16. A composition comprising a surrogate mRNA drug product comprised of a physical property surrogate, a chemical property surrogate and/or a visual property surrogate, wherein the surrogate mRNA drug product has at least one quantitative or qualitative property selected from the group consisting of viscosity, turbidity, density, and surface tension that is substantially the same as a mRNA drug product comprised of mRNA in a LNP, and wherein the surrogate mRNA drug product does not comprise mRNA or LNP.

17. A method for assessing a physical, chemical and/or visual property of an LNP and/or mRNA drug product, comprising
identifying a physical, chemical and/or a visual property of the composition of any one of the preceding claims, and
determining that the physical, chemical and/or visual property of the LNP and/or mRNA drug product is within a pharmaceutically acceptable range.

18. A method of calibrating an instrument for determining a physical, chemical and/or visual property of a LNP and/or mRNA drug product, the method comprising performing a measurement on the composition of any one of claims 1-16 with the instrument, and optionally modifying one or more settings of the instrument.

19. The method of claim 18, further comprising measuring the physical, chemical and/or visual property of the mRNA drug product.

20. The method of claim 18 or 19, wherein the instrument measures viscosity, density, surface tension, clarity, or hue.

21. A container comprising a vial or syringe, wherein the container houses a surrogate mRNA drug product comprised of a physical property surrogate, a chemical property surrogate and/or a visual property surrogate and wherein the container does not comprise a lipid nanoparticle (LNP) or mRNA.

22. The container of claim 21, wherein the surrogate mRNA drug product comprises the composition of any one of claims 1-16.
